(19) United States
(12) Patent Application Publication
(10) Pub. No.: US 2013/0172304 A1
(43) Pub. Date: Jul. 4, 2013

(54) Pharmaceutical Compositions Comprising Flibanserin and a Further Agent in the Treatment of Sexual Disorders

(71) Applicant: SPROUT PHARMACEUTICALS INC., Raleigh, NC (US)

(72) Inventor: Georg Boeck, Laupheim (DE)

(73) Assignee: SPROUT PHARMACEUTICALS INC., Raleigh, NC (US)

(21) Appl. No.: 13/749,152

(22) Filed: Jan. 24, 2013

Related U.S. Application Data
(63) Continuation of application No. 12/532,269, filed on Dec. 14, 2009, now abandoned.

(30) Foreign Application Priority Data
Mar. 28, 2007 (EP) 07105104.9

(51) Int. Cl. A61K 31/573; A61K 45/06; A61K 31/57; A61K 31/496; A61K 31/565

(52) U.S. Cl. CPC A61K 31/573; A61K 31/496; A61K 31/565; A61K 31/57; A61K 45/06
USPC 514/171; 514/254.06

(57) ABSTRACT

The invention relates to new pharmaceutical compositions for the treatment of sexual disorders and methods for the preparation thereof. In a preferred embodiment, the instant invention is directed to pharmaceutical combinations comprising flibanserin as one active ingredient in combination with at least one additional active ingredient selected from the group consisting of compounds used for female hormone replacement therapy 2a, compounds used for menopause problems 2b, and contraceptives 2c.

The invention relates to new pharmaceutical combinations comprising flibanserin as one active ingredient in combination with at least one additional active ingredient for the treatment of sexual disorders and methods for the preparation thereof.

### BACKGROUND OF THE INVENTION

The instant invention is directed to pharmaceutical combinations comprising a therapeutically effective amount of flibanserin 1 as one active ingredient in combination with a therapeutically effective amount of at least one additional active ingredient 2 selected from the group consisting of compounds used for female hormone replacement therapy 2a, compounds used for menopause problems 2b, and contraceptives 2c, for the treatment of sexual disorders and methods for the preparation thereof.

The compound 1-[2-(4-(3-trifluoromethyl-phenyl)piperazin-1-yl)ethyl]-2,3-dihydro-1H-benzimidazol-2-one (flibanserin) is disclosed in form of its hydrochloride in European Patent Application EP-A-526434 and has the following chemical structure:

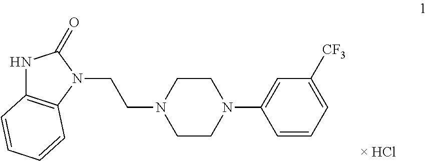 [SMILES: O=c1[nH]c2ccccc2n1CCN1CCN(c2cccc(C(F)(F)F)c2)CC1]

Flibanserin shows affinity for the 5-HT_{1A} and 5-HT₂-receptor. It is therefore a promising therapeutic agent for the treatment of a variety of diseases, for instance depression, schizophrenia, Parkinson, anxiety, sleep disturbances, sexual and mental disorders and age associated memory impairment.

One embodiment of the invention is directed to pharmaceutical compositions comprising a therapeutically effective amount of flibanserin 1 in combination with a therapeutically effective amount of one or more, preferably one active ingredient 2 selected from the group consisting of compounds used for female hormone replacement therapy 2a, compounds used for menopause problems 2b, and contraceptives 2c.

A preferred embodiment of the present invention relates to pharmaceutical compositions comprising a therapeutically effective amount of flibanserin 1 in combination with a therapeutically effective amount of one or more, preferably one contraceptive 2c.

The compositions according to the invention may contain flibanserin 1 and the one or more additional active ingredient 2 in a single formulation or in separate formulations. If flibanserin and the one or more additional active ingredient are present in separate formulations these separate formulations may be administered simultaneously or sequentially.

The combinations of the present invention are especially useful for female patients who are in need of hormone replacement therapy, female patients with menopause problems and females receiving contraceptives and, in addition, suffer from sexual diseases. By combination of flibanserin with those other drugs, dosing is simplified, is more convenient and therefore leads to better compliance with therapy.

A preferred embodiment according to the invention is directed to pharmaceutical compositions comprising a therapeutically effective amount of flibanserin 1 and a therapeutically effective amount of one or more, preferably one compound used for female hormone replacement therapy 2a, optionally in combination with a pharmaceutically acceptable excipient. Examples of suitable compounds used for female hormone replacement therapy 2a include chlormadinone acetate, dienogest, dydrogesterone, estradiole valerate, medroxyprogesterone, medroxyprogesterone acetate, norethisterone and norethisterone acetate, optionally in form of the pharmaceutically acceptable acid addition salts, in form of the hydrates and/or solvates and optionally in the form of the individual optical isomers, mixtures of the individual enantiomers or racemates thereof.

Another preferred embodiment according to the invention is directed to pharmaceutical compositions comprising a therapeutically effective amount of flibanserin 1 and a therapeutically effective amount of one or more, preferably one compound used for menopause problems 2b, optionally in combination with a pharmaceutically acceptable excipient. Examples of suitable compounds used for menopause problems 2b include estradiole valerate, medroxyprogesterone acetate, norgestrel, prasteronenantat, and progesterone, optionally in form of the pharmaceutically acceptable salts, in form of the hydrates and/or solvates and optionally in the form of the individual optical isomers, mixtures of the individual enantiomers or racemates thereof.

A more preferred embodiment according to the invention is directed to pharmaceutical compositions comprising a therapeutically effective amount of flibanserin 1 and a therapeutically effective amount of one or more, preferably one contraceptive 2c, optionally in combination with a pharmaceutically acceptable excipient. Examples of suitable contraceptives 2c include chlormadinone acetate, dienogest, drospirenone, etonogestrel, gestodene, medroxyprogesterone acetate, norelgestromine, norethisterone, norethisterone enantate, and norgestimate, optionally in form of the pharmaceutically acceptable salts, in form of the hydrates and/or solvates and optionally in the form of the individual optical isomers, mixtures of the individual enantiomers or racemates thereof.

Flibanserin 1 may be used in form of the free base, optionally in form of its pharmaceutically acceptable acid addition salts and/or optionally in form of the hydrates and/or solvates thereof. Suitable acid addition salts include for example those of the acids selected from, succinic acid, hydrobromic acid, acetic acid, fumaric acid, maleic acid, methanesulphonic acid, lactic acid, phosphoric acid, hydrochloric acid, sulphuric acid, tartaric acid and citric acid. Mixtures of the abovementioned acid addition salts may also be used. From the aforementioned acid addition salts the hydrochloride and the hydrobromide, particularly the hydrochloride, are preferred. If flibanserin 1 is used in form of the free base, it is preferably used in form of flibanserin polymorph A as disclosed in WO 03/014079.

The active ingredients 2 which are suitable to be combined with flibanserin within the teaching of the instant invention and which are mentioned hereinbefore may also be capable of forming acid addition salts with pharmaceutically acceptable acids. Representative salts include the following: Acetate, Benzenesulfonate, Benzoate, Bicarbonate, Bisulfate, Bitartrate, Borate, Bromide, Camsylate, Carbonate, Chloride, Clavulanate, Citrate, Dihydrochloride, Edetate, Edisylate, Estolate, Esylate, Fumarate, Gluceptate, Gluconate, Glutamate, Glycollylarsanilate, Hexylresorcinate, Hydrabamine, Hydrobromide, Hydrochloride, Hydroxynaphthoate, Iodide, Isothionate, Lactate, Lactobionate, Laurate, Malate, Maleate, Mandelate, Mesylate, Methylbromide, Methylnitrate, Methylsulfate, Mucate, Napsylate, Nitrate, N-methylglucamine ammonium salt, Oleate, Oxalate, Pamoate (Embonate), Palmitate, Pantothenate, Phosphate/diphosphate, Polygalacturonate, Salicylate, Stearate, Sulfate, Subacetate, Succinate, Tannate, Tartrate, Teoclate, Tosylate, Triethiodide and Valerate.

Furthermore, where the compounds 2 carry an acidic moiety, suitable pharmaceutically acceptable salts thereof may include alkali metal salts, e.g., sodium or potassium salts; alkaline earth metal salts, e.g., calcium or magnesium salts; and salts formed with suitable organic ligands, e.g., quaternary ammonium salts.

The compounds 2 may have chiral centers and occur as racemates, racemic mixtures and as individual diastereomers, or enantiomers with all isomeric forms being included in the present invention. Therefore, where a compound is chiral, the separate enantiomers, substantially free of the other, are included within the scope of the invention. Further included are all mixtures of the two enantiomers. Also included within the scope of the invention are polymorphs and hydrates of the compounds of the instant invention.

The present invention includes within its scope prodrugs of the compounds 1 and 2. In general, such prodrugs will be functional derivatives of the compounds of this invention which are readily convertible in vivo into the required compound.

The term “therapeutically effective amount” shall mean that amount of a drug or pharmaceutical agent that will elicit the biological or medical response of a tissue, system, animal or human that is being sought by a researcher or clinician.

As used herein, the term “composition” is intended to encompass a product comprising the specified ingredients in the specified amounts, as well as any product which results, directly or indirectly, from combination of the specified ingredients in the specified amounts.

In the combination of the present invention, the components 1 and 2 may be administered separately or together in one pharmaceutical composition. In addition, the administration of one element of the combination of the present invention may be prior to, concurrent to, or subsequent to the administration of the other element of the combination.

The elements of the combination of 1 and 2 may be administered by oral, parenteral (e.g., intramuscular, intraperitoneal, intravenous or subcutaneous injection, or implant), buccal, nasal, vaginal, rectal, sublingual, or topical (e.a. ocular eyedrop) routes of administration and may be formulated, alone or together, in suitable dosage unit formulations containing conventional non-toxic pharmaceutically acceptable carriers, adjuvants and vehicles appropriate for each route of administration.

The pharmaceutical compositions for the administration of the components 1 and 2 of this invention may conveniently be presented in dosage unit form and may be prepared by any of the methods well known in the art of pharmacy. All methods include the step of bringing the active ingredient into association with the carrier which is constituted of one or more accessory ingredients. In general, the pharmaceutical compositions are prepared by uniformly and intimately bringing the active ingredients into association with a liquid carrier or a finely divided solid carrier or both, and then, if necessary, shaping the product into the desired dosage form. In the pharmaceutical compositions the active compounds are included in an amount sufficient to produce the desired pharmacologic effect.

The pharmaceutical compositions containing the active ingredients 1 and 2, separately or together, that are suitable for oral administration may be in the form of discrete units such as hard or soft capsules, tablets, troches or lozenges, each containing a predetermined amount of the active ingredients; in the form of a dispersible powder or granules; in the form of a solution or a suspension in an aqueous liquid or non-aqueous liquid; in the form of syrups or elixirs; or in the form of an oil-in-water emulsion or a water-in-oil emulsion.

Dosage forms intended for oral use may be prepared according to any method known to the art for the manufacture of pharmaceutical formulations and such compositions.

The excipients used may be for example, (a) inert diluents such as mannitol, sorbitol, calcium carbonate, pregelatinized starch, lactose, calcium phosphate or sodium phosphate; (b) granulating and disintegrating agents, such as povidone, copovidone, hydroxypropylmethylcellulose, corn starch, alginic acid, crospovidone, sodiumstarchglycolate, croscarmellose, or polacrilin potassium; (c) binding agents such as microcrystalline cellulose or acacia; and (d) lubricating agents such as magnesium stearate, stearic acid, fumaric acid or talc.

In some cases, formulations for oral use may be in the form of hardgelatin or HPMC capsules wherein the active ingredient 1 or 2, separately or together, is mixed with an inert solid diluent, for example pregelatinized starch, calcium carbonate, calcium phosphate or kaolin, or dispensed via a pellet formulation. They may also be in the form of soft gelatin capsules wherein the active ingredient is mixed with water or an oil medium, for example peanut oil, liquid paraffin, medium chain triglycerides or olive oil.

The tablets, capsules or pellets may be uncoated or they may be coated by known techniques to delay disintegration and absorption in the gastrointestinal tract and thereby provide a delayed action or sustained action over a longer period. For example, a time delay material such as celluloseacetate phtalate or hydroxypropylcellulose acetate succinate or sustained release material such as ethylcellulose or ammoniomethacrylate copolymer (type B) may be employed.

Liquid dosage forms for oral administration include pharmaceutically acceptable emulsions, solutions, suspensions, syrups, and elixirs containing inert diluents commonly used in the art, such as water. Besides such inert diluents, compositions can also include adjuvants, such as wetting agents, emulsifying and suspending agents, and sweetening, flavoring, perfuming and preserving agents.

Aqueous suspensions normally contain the active materials 1 and 2, separately or together, in admixture with excipients suitable for the manufacture of aqueous suspensions. Such excipients may be (a) suspending agents such as hydroxy ethylcellulose, sodium carboxymethylcellulose, methylcellulose, hydroxypropylmethylcellulose, sodium alginate, polyvinylpyrrolidone, gum tragacanth and gum acacia; (b) dispersing or wetting agents which may be (b.1) a naturally-occurring phosphatide such as lecithin, (b.2) a condensation product of an alkylene oxide with a fatty acid, for example, polyoxyethylene stearate, (b.3) a condensation product of ethylene oxide with a long chain aliphatic alcohol, for example heptadecaethyleneoxycetanol, (b.4) a condensation product of ethylene oxide with a partial ester derived from a fatty acid and a hexitol such as polyoxyethylene sorbitol monooleate, or (b.5) a condensation product of ethylene oxide with a partial ester derived from a fatty acid and a hexitol anhydride, for example polyoxyethylene sorbitan monooleate.

The aqueous suspensions may also contain one or more preservatives, for example, ethyl or n-propyl p-hydroxybenzoate; one or more coloring agents; one or more flavoring agents; and one or more sweetening agents, such as sucrose or saccharin.

Oily suspensions may be formulated by suspending the active ingredients 1 and 2, separately or together, in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol.

Sweetening agents and flavoring agents may be added to provide a palatable oral preparation. These compositions may be prepared by the addition of an antioxidant such as ascorbic acid.

Dispersible powders and granules are suitable for the preparation of an aqueous suspension. They provide the active ingredients 1 and 2, separately or together, in admixture with a dispersing or wetting agent, a suspending agent and one or more preservatives. Suitable dispersing or wetting agents and suspending agents are exemplified by those already mentioned above. Additional excipients, for example, those sweetening, flavoring and coloring agents described above may also be present.

The pharmaceutical compositions of the invention may also be in the form of oil-in-water emulsions. The oily phase may be a vegetable oil such as olive oil or arachis oils, or a mineral oil such as liquid paraffin or a mixture thereof.

Suitable emulsifying agents may be (a) naturally-occurring gums such as gum acacia and gum tragacanth, (b) naturally-occurring phosphatides such as soybean and lecithin, (c) esters or partial esters derived from fatty acids and hexitol anhydrides, for example, sorbitan monooleate, (d) condensation products of said partial esters with ethylene oxide, for example polyoxyethylene sorbitan monooleate. The emulsions may also contain sweetening and flavoring agents.

Syrups and elixirs may be formulated with sweetening agents, for example, glycerol, propylene glycol, sorbitol or sucrose. Such formulations may also contain a preservative and flavoring and coloring agents.

The pharmaceutical compositions containing 1 and 2, separately or together, may be in the form of a sterile injectable aqueous or oleagenous suspension or solution. The suspension may be formulated according to known methods using those suitable dispersing or wetting agents and suspending agents which have been mentioned above. The sterile injectable preparation may also be a sterile injectable solution or suspension in a non toxic parenterally-acceptable diluent or solvent, for example as a solution in 1,3-butane-diol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

Preparations according to this invention containing 1 and 2, separately or together, for parenteral administration include sterile aqueous or non-aqueous solutions, suspension, or emulsions.

Examples of non-aqueous solvents or vehicles are propylene glycol, polyethylene glycol, vegetable oils, such as olive oil and corn oil, gelatin, and injectable organic esters such as ethyl oleate. Such dosage forms may also contain adjuvants such as preserving, wetting, emulsifying, and dispersing agents. They may be sterilized by, for example, filtration through a bacteria-retaining filter, by incorporating sterilizing agents into the compositions, by irradiating the compositions, or by heating the compositions. They can also be manufactured in the form of sterile solid compositions which can be reconstituted in sterile water, or some other sterile injectable medium immediately before use. The combination of this invention may also be administered in the form of suppositories for rectal administration. This composition can be prepared by mixing the drugs with a suitable non-irritating excipient which is solid at ordinary temperatures but liquid at the rectal temperature and will therefore melt in the rectum to release the drug. Such materials are cocoa butter, hard fat, and polyethylene glycols. Compositions for buccal, nasal or sublingual administration are also prepared with standard excipients well known in the art.

For topical administration the combinations of this invention containing 1 and 2, separately or together, may be formulated in liquid or semi-liquid preparations such as liniments, lotions, applications; oil-in-water or water-in-oil emulsions such as creams, ointments, jellies or pastes, including tooth-pastes; or solutions or suspensions such as drops, and the like.

The dosage of the active ingredients in the compositions of this invention may be varied. However, it is necessary that the amount of the active ingredients 1 and 2 be such that a suitable dosage form is obtained. The selected dosage and the dosage form depend upon the desired therapeutic effect, on the route of administration and on the duration of the treatment. Dosage ranges in the combination are approximately one tenth to one times the clinically effective ranges required to induce the desired therapeutic effect, respectively when the compounds are used singly.

Within the instant invention flibanserin 1 is preferably administered in such an amount that per single dosage between 5 to 200 mg of flibanserin 1 are applied. Preferred are ranges of between 10 to 150 mg, particular preferred 20 to 100 mg of flibanserin 1. Suitable dosage forms may contain for instance 20, 25, 30, 35, 40, 45, 50, 55, 60, 65, 70, 75, 80, 85, 90, 95 or 100 mg of flibanserin 1. The aforementioned values are based on flibanserin 1 in form of the free base. If flibanserin 1 is applied in form of one of its acid addition salts, the corresponding values are readily calculable from the aforementioned values.

Within the instant invention the compounds used for female hormone replacement therapy 2a are preferably administered in a range of between about 0.001 mg per kg of bodyweight per day (mg/kg/day) to about 100 mg/kg/day, preferably 0.01 to 50 mg/kg/day, and most preferably 0.1 to 30 mg/kg/day. Intravenously, the most preferred doses will range from about 0.1 to about 10 mg/kg/minute during a constant rate infusion. Advantageously, the compounds 2a of the present invention may be administered in a single daily dose, or the total daily dosage may be administered in divided doses of two, three or four times daily. Suitable dosage forms may contain for instance 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 1.05, 1.1, 1.15, 1.2, 1.25, 1.3, 1.35, 1.4, 1.45, 1.5, 1.55, 1.6, 1.65, 1.7, 1.75, 1.8, 1.85, 1.9, 1.95, 2, 2.05, 2.1, 2.15, 2.2, 2.25, 2.3, 2.35, 2.4, 2.45, 2.5, 2.55, 2.6, 2.65, 2.7, 2.75, 2.8, 2.85, 2.9, 2.95, 3, 3.05, 3.1, 3.15, 3.2, 3.25, 3.3, 3.35, 3.4, 3.45, 3.5, 3.55, 3.6, 3.65, 3.7, 3.75, 3.8, 3.85, 3.9, 3.95, 4, 4.05, 4.1, 4.15, 4.2, 4.25, 4.3, 4.35, 4.4, 4.45, 4.5, 4.55, 4.6, 4.65, 4.7, 4.75, 4.8, 4.85, 4.9, 4.95, 5, 10, 15, 20, 25, 30, 35, 40, 45, 50, 55, 60, 65, 70, 75, 80, 85, 90, 95, or 100 mg of 2a. Advantageously, the compounds 2a of the present invention may be administered in a single daily dose, or the total daily dosage may be administered in divided doses of two, three or four times daily.

Within the instant invention the compounds used for menopause problems 2b are preferably administered in a range of between about 0.001 mg per kg of bodyweight per day (mg/kg/day) to about 100 mg/kg/day, preferably 0.01 to 50 mg/kg/day, and most preferably 0.1 to 10 mg/kg/day. Intravenously, the most preferred doses will range from about 0.1 to about 10 mg/kg/minute during a constant rate infusion. Advantageously, the compounds 2b of the present invention may be administered in a single daily dose, or the total daily dosage may be administered in divided doses of two, three or four times daily. Suitable dosage forms may contain for instance 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 1.05, 1.1, 1.15, 1.2, 1.25, 1.3, 1.35, 1.4, 1.45, 1.5, 1.55, 1.6, 1.65, 1.7, 1.75, 1.8, 1.85, 1.9, 1.95, 2, 2.05, 2.1, 2.15, 2.2, 2.25, 2.3, 2.35, 2.4, 2.45, 2.5, 2.55, 2.6, 2.65, 2.7, 2.75, 2.8, 2.85, 2.9, 2.95, 3, 3.05, 3.1, 3.15, 3.2, 3.25, 3.3, 3.35, 3.4, 3.45, 3.5, 3.55, 3.6, 3.65, 3.7, 3.75, 3.8, 3.85, 3.9, 3.95, 4, 4.05, 4.1, 4.15, 4.2, 4.25, 4.3, 4.35, 4.4, 4.45, 4.5, 4.55, 4.6, 4.65, 4.7, 4.75, 4.8, 4.85, 4.9, 4.95, 5, 10, 15, 20, 25, 30, 35, 40, 45, 50, 55, 60, 65, 70, 75, 80, 85, 90, 95, or 100 mg of 2b. Advantageously, the compounds 2b of the present invention may be administered in a single daily dose, or the total daily dosage may be administered in divided doses of two, three or four times daily.

Within the instant invention the contraceptives 2c are preferably administered in a range of between about 0.001 mg per kg of bodyweight per day (mg/kg/day) to about 100 mg/kg/day, preferably 0.01 to 10 mg/kg/day, and most preferably 0.1 to 1 mg/kg/day. Advantageously, the compounds 2c of the present invention may be administered in a single daily dose, or the total daily dosage may be administered in divided doses of two, three or four times daily. Suitable dosage forms may contain for instance 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 1.05, 1.1, 1.15, 1.2, 1.25, 1.3, 1.35, 1.4, 1.45, 1.5, 1.55, 1.6, 1.65, 1.7, 1.75, 1.8, 1.85, 1.9, 1.95, 2, 2.05, 2.1, 2.15, 2.2, 2.25, 2.3, 2.35, 2.4, 2.45, 2.5, 2.55, 2.6, 2.65, 2.7, 2.75, 2.8, 2.85, 2.9, 2.95, 3, 3.05, 3.1, 3.15, 3.2, 3.25, 3.3, 3.35, 3.4, 3.45, 3.5, 3.55, 3.6, 3.65, 3.7, 3.75, 3.8, 3.85, 3.9, 3.95, 4, 4.05, 4.1, 4.15, 4.2, 4.25, 4.3, 4.35, 4.4, 4.45, 4.5, 4.55, 4.6, 4.65, 4.7, 4.75, 4.8, 4.85, 4.9, 4.95, 5, 10, 15, 20, 25, 30, 35, 40, 45, 50, 55, 60, 65, 70, 75, 80, 85, 90, 95, or 100 mg of 2c. Advantageously, the compounds 2c of the present invention may be administered in a single daily dose, or the total daily dosage may be administered in divided doses of two, three or four times daily.

However, it should be apparent to one skilled in the art that the exact dosage and frequency of administration will depend on the particular compounds of the invention administered, the particular condition being treated, the severity of the condition being treated, the age, weight, general physical condition of the particular patient, and other medication the individual may be taking as is well known to administering physicians who are skilled in this art.

In another embodiment the invention relates to a method for the treatment of sexual disorders, comprising the administration of a therapeutically effective amount of flibanserin 1, in combination with a therapeutically effective amount of 2, selected from the group consisting of compounds used for female hormone replacement therapy 2a, compounds used for menopause problems 2b, and contraceptives 2c, separately or together within one pharmaceutical composition.

The generic term “Sexual disorders” includes Sexual Desire Disorders (i.e. Hypoactive Sexual Desire Disorder, Sexual Aversion Disorder), Sexual Arousal Disorders (i.e. Female Sexual Arousal Disorder, Male Erectile Disorder), Orgasmic Disorders (i.e. Female Orgasmic Disorder, Male Orgasmic Disorder, Premature Ejaculation) Sexual Pain Disorders (i.e. Dyspareunia, Vaginismus), Sexual Dysfunction due to a General Medical Condition, Substance-Induced Sexual Dysfunction, and Sexual Dysfunction not otherwise specified (Diagnostic and Statistical Manual of Mental Disorders, 4th edition, Text Revision. Washington D.C., American Psychiatric Association, 2000).

In another preferred embodiment the invention relates to a method for the treatment of disorders selected from the group consisting of hypoactive sexual desire disorder (HSDD), sexual aversion disorder, loss of sexual desire, lack of sexual desire, decreased sexual desire, inhibited sexual desire, loss of libido, libido disturbance, and frigidity, comprising the administration of a therapeutically effective amount of flibanserin 1, in combination with a therapeutically effective amount of 2, optionally in form of the pharmaceutically acceptable acid addition salts, in form of the hydrates and/or solvates and optionally in the form of the individual optical isomers, mixtures of the individual enantiomers or racemates thereof, separately or together within one pharmaceutical composition.

In another preferred embodiment the invention relates to a method for the treatment of disorders selected from the group consisting of hypoactive sexual desire disorder (HSDD), sexual aversion disorder, loss of sexual desire, lack of sexual desire, decreased sexual desire, inhibited sexual desire, comprising the administration of a therapeutically effective amount of flibanserin 1, in combination with a therapeutically effective amount of 2, optionally in form of the pharmaceutically acceptable acid addition salts, in form of the hydrates and/or solvates and optionally in the form of the individual optical isomers, mixtures of the individual enantiomers or racemates thereof, separately or together within one pharmaceutical composition.

In another preferred embodiment the invention relates to a method for treatment of disorders selected from the group of hypoactive sexual desire disorder (HSDD), decreased sexual desire and inhibited sexual desire, comprising the administration of a therapeutically effective amount of flibanserin 1, in combination with a therapeutically effective amount of 2, optionally in form of the pharmaceutically acceptable acid addition salts, in form of the hydrates and/or solvates and optionally in the form of the individual optical isomers, mixtures of the individual enantiomers or racemates thereof, separately or together within one pharmaceutical composition.

In another preferred embodiment the invention relates to a method for treatment of premenstrual disorders, comprising the administration of a therapeutically effective amount of flibanserin 1, in combination with a therapeutically effective amount of 2, optionally in form of the pharmaceutically acceptable acid addition salts, in form of the hydrates and/or solvates and optionally in the form of the individual optical isomers, mixtures of the individual enantiomers or racemates thereof, separately or together within one pharmaceutical composition.

In another preferred embodiment the invention relates to a method for treatment of premenstrual disorders selected from the group consisting of premenstrual dysphoria, premenstrual syndrome, premenstrual dysphoric disorder, comprising the administration of a therapeutically effective amount of flibanserin 1, in combination with a therapeutically effective amount of 2, optionally in form of the pharmaceutically acceptable acid addition salts, in form of the hydrates and/or solvates and optionally in the form of the individual optical isomers, mixtures of the individual enantiomers or racemates thereof, separately or together within one pharmaceutical composition.

In another preferred embodiment the invention relates to a method for treatment of sexual arousal disorder in females, comprising the administration of a therapeutically effective amount of flibanserin 1, in combination with a therapeutically effective amount of 2, optionally in form of the pharmaceutically acceptable acid addition salts, in form of the hydrates and/or solvates and optionally in the form of the individual optical isomers, mixtures of the individual enantiomers or racemates thereof, separately or together within one pharmaceutical composition.

In another preferred embodiment the invention relates to a method for treatment of orgasmic disorder in females, comprising the administration of a therapeutically effective amount of flibanserin 1, in combination with a therapeutically effective amount of 2, optionally in form of the pharmaceutically acceptable acid addition salts, in form of the hydrates and/or solvates and optionally in the form of the individual optical isomers, mixtures of the individual enantiomers or racemates thereof, separately or together within one pharmaceutical composition.

In another preferred embodiment the invention relates to a method for treatment of sexual pain disorders in females, comprising the administration of a therapeutically effective amount of flibanserin 1, in combination with a therapeutically effective amount of 2, optionally in form of the pharmaceutically acceptable acid addition salts, in form of the hydrates and/or solvates and optionally in the form of the individual optical isomers, mixtures of the individual enantiomers or racemates thereof, separately or together within one pharmaceutical composition.

In another preferred embodiment the invention relates to a method for treatment of sexual pain disorders selected from the group consisting of dyspareunia in females, vaginismus in females, and noncoital sexual pain disorder in females, comprising the administration of a therapeutically effective amount of flibanserin 1, in combination with a therapeutically effective amount of 2, optionally in form of the pharmaceutically acceptable acid addition salts, in form of the hydrates and/or solvates and optionally in the form of the individual optical isomers, mixtures of the individual enantiomers or racemates thereof, separately or together within one pharmaceutical composition.

In another preferred embodiment the invention relates to a method for treatment of sexual dysfunction due to a general medical condition in females, comprising the administration of a therapeutically effective amount of flibanserin 1, in combination with a therapeutically effective amount of 2, optionally in form of the pharmaceutically acceptable acid addition salts, in form of the hydrates and/or solvates and optionally in the form of the individual optical isomers, mixtures of the individual enantiomers or racemates thereof, separately or together within one pharmaceutical composition.

In another preferred embodiment the invention relates to a method for treatment of substance-induced sexual dysfunction in females, comprising the administration of a therapeutically effective amount of flibanserin 1, in combination with a therapeutically effective amount of 2, optionally in form of the pharmaceutically acceptable acid addition salts, in form of the hydrates and/or solvates and optionally in the form of the individual optical isomers, mixtures of the individual enantiomers or racemates thereof, separately or together within one pharmaceutical composition.

The beneficial effects of the compounds of formula (I) can be observed regardless of whether the above mentioned sexual disorders existed lifelong or was acquired, is of the “generalized type” or “situational type” and independent of etiologic origin (organic—both, physically and drug induced-, psychogen (due to psychological factors), a combination of organic—both, physically and drug induced-, and psychogen (due to combined factors), or unknown) origin. The term “lifelong” refers to such sexual disorders of the present invention, which have been present since the onset of sexual functioning. The term “acquired” refers to such sexual disorders of the present invention which developed only after a period of normal sexual functioning. The “generalized type” refers to such sexual disorders of the present invention wherein the disorder is not limited to certain types of stimulation, situations, or partners. The “situational type” applies to such sexual disorders of the present invention wherein the disorder is limited to certain types of stimulation, situations, or partners. The subtype due to “psychological factors” applies when psychological factors are judged to have the major role in the onset, severity, exacerbation, or maintenance of the Sexual Disorder, and general medical conditions and substance play no role in the etiology of the sexual disorder. Finally the subtype due to “combined factors” applies when 1) psychological factors are judged to have a role in the onset, severity, exacerbation, or maintenance of the sexual disorder, and 2) a general medical condition or substance use is also judged to be contributory but is not sufficient to account for a Sexual Disorder (Diagnostic and Statistical Manual of Mental Disorders, 4th edition, Text Revision. Washington D.C., American Psychiatric Association, 2000).

The beneficial effects of the compounds of formula (I) can also be observed regardless of whether the females suffering from above mentioned diseases are in the pre-menopausal, peri-menopausal or post-menopausal state.

Another embodiment of the present invention relates to the use of the combinations of a therapeutically effective amount of flibanserin 1, and of a therapeutically effective amount of 2, optionally in form of the pharmaceutically acceptable salts, in form of the hydrates and/or solvates and optionally in the form of the individual optical isomers, mixtures of the individual enantiomers or racemates thereof, for the manufacture of a medicament for the treatment of any of the aforementioned disorders.

Another embodiment of the present invention relates to the use of a therapeutically effective amount of flibanserin 1, for the manufacture of a medicament for the treatment of any of the aforementioned disorders in combination with a therapeutically effective amount of 2, optionally in form of the pharmaceutically acceptable salts, in form of the hydrates and/or solvates and optionally in the form of the individual optical isomers, mixtures of the individual enantiomers or racemates thereof.

In a preferred embodiment, the invention relates to any of the above mentioned methods and uses wherein the therapeutically effective amount of 2 is selected from compounds used for female hormone replacement therapy 2a.

In a more preferred embodiment, the invention relates to any of the above mentioned methods and uses wherein the therapeutically effective amount of compounds used for female hormone replacement therapy 2a are selected from the group consisting of chlormadinone acetate, dienogest, dydrogesterone, estradiole valerate, medroxyprogesterone, medroxyprogesterone acetate, norethisterone and norethisterone acetate.

In a preferred embodiment, the invention relates to any of the above mentioned methods and uses wherein the therapeutically effective amount of 2 is selected from compounds used for menopause problems 2b.

In a more preferred embodiment, the invention relates to any of the above mentioned methods and uses wherein the therapeutically effective amount of 2 is selected from compounds used for menopause problems 2b are selected from the group consisting of estradiole valerate, medroxyprogesterone acetate, norgestrel, prasteronenantat, and progesterone.

In a preferred embodiment, the invention relates to any of the above mentioned methods and uses wherein the therapeutically effective amount of 2 is selected from contraceptives 2c.

In a more preferred embodiment, the invention relates to any of the above mentioned methods and uses wherein the therapeutically effective amount of 2 is selected from contraceptives 2c are selected from the group consisting of chlormadinone acetate, dienogest, drospirenone, etonogestrel, gestodene, medroxyprogesterone acetate, norelgestromine, norethisterone, norethisterone enantate, and norgestimate.

The following examples demonstrate possible pharmaceutical compositions comprising flibanserin in combination with one of the aforementioned combination partners 2.

### EXAMPLE NO. 1

### Combination 1

| | |
|---|---|
| Constituents | mg/tablet |

Core

| | |
|---|---|
| Flibanserin (free base) | 50.000 |
| Dienogest | 2.000 |
| Anhydrous dibasic calcium phosphate | 100.000 |
| Microcrystalline cellulose | 203.090 |
| HPMC (Methocel E5) | 6.615 |
| Croscarmellose sodium | 8.820 |
| Magnesium stearate | 2.250 |

Coating

| | |
|---|---|
| HPMC (Methocel E5) | 4.320 |
| Polyethylene Glycol 6000 | 1.260 |
| Titanium dioxide | 1.800 |
| Talc | 1.542 |
| Iron oxide red | 0.078 |
| Total Film coated tablet | 381.775 |

### EXAMPLE NO. 2

### Combination 2

| | |
|---|---|
| Constituents | mg/tablet |

Core

| | |
|---|---|
| Flibanserin (free base) | 50.000 |
| Estradiole valerate | 2.000 |
| Lactose monohydrate | 133.750 |
| Microcrystalline cellulose | 40.000 |
| Hydroxypropylcellulose | 2.500 |
| Corn starch | 12.500 |
| Magnesium stearate | 1.250 |

Coating

| | |
|---|---|
| HPMC (e.g. Pharmacoat 606) | 2.400 |
| Polyethylene Glycol 6000 | 0.700 |
| Titanium dioxide | 1.000 |
| Talc | 0.857 |
| Iron oxide yellow | 0.043 |
| Total Film coated tablet | 247.000 |

### EXAMPLE NO. 3

### Combination 3

| | |
|---|---|
| Constituents | mg/tablet |

Core

| | |
|---|---|
| Flibanserin (free base) | 50.000 |
| Chlormadinone acetate | 1.000 |
| Lactose monohydrate | 143.490 |
| Microcrystalline cellulose | 47.810 |
| HPMC (e.g. Pharmacoat 606) | 2.500 |
| Carboxymethylcellulose sodium | 5.000 |
| Mannitol | 60.000 |
| Corn starch | 36.500 |
| Povidone | 1.000 |
| Colloidal silicon dioxide | 1.000 |
| Magnesium stearate | 1.700 |

Coating

| | |
|---|---|
| HPMC (e.g. Methocel E5) | 3.360 |
| Polyethylene Glycol 6000 | 0.980 |
| Titanium dioxide | 1.400 |
| Talc | 1.200 |
| Iron oxide red | 0.060 |
| Total Film coated bilayer tablet | 357.000 |

The following examples show preferred pharmaceutical compositions of flibanserin, if the combinations according to the invention are administered in separate dosage units.

### EXAMPLE NO. 4

### Composition

| | |
|---|---|
| Constituents | mg/tablet |

Core

| | |
|---|---|
| Flibanserin (free base) | 25.000 |
| Lactose monohydrate | 71.720 |
| Microcrystalline cellulose | 23.905 |
| HPMC (Methocel E5) | 1.250 |
| Carboxymethylcellulose sodium | 2.500 |
| Magnesium stearate | 0.625 |

Coating

| | |
|---|---|
| HPMC (Methocel E5) | 1.440 |
| Polyethylene Glycol 6000 | 0.420 |
| Titanium dioxide | 0.600 |
| Talc | 0.514 |
| Iron oxide red | 0.026 |
| Total Film coated tablet | 128.000 |

### EXAMPLE NO. 5

### Composition

| | |
|---|---|
| Constituents | mg/tablet |

Core

| | |
|---|---|
| Flibanserin (free base) | 50.000 |
| Lactose monohydrate | 143.440 |
| Microcrystalline cellulose | 47.810 |
| HPMC (e.g. Pharmacoat 606) | 2.500 |
| Carboxymethylcellulose sodium | 5.000 |
| Magnesium stearate | 1.250 |

Coating

| | |
|---|---|
| HPMC (e.g. Pharmacoat 606) | 2.400 |
| Polyethylene Glycol 6000 | 0.700 |
| Titanium dioxide | 1.000 |
| Talc | 0.857 |
| Iron oxide red | 0.043 |
| Total Film coated tablet | 255.000 |

### EXAMPLE NO. 6

### Composition

| | |
|---|---|
| Constituents | mg/tablet |

Core

| | |
|---|---|
| Flibanserin (free base) | 100.000 |
| Lactose monohydrate | 171.080 |
| Microcrystalline cellulose | 57.020 |
| HPMC (e.g. Methocel E5) | 3.400 |
| Carboxymethylcellulose sodium | 6.800 |
| Magnesium stearate | 1.700 |

Coating

| | |
|---|---|
| HPMC (e.g. Methocel E5) | 3.360 |
| Polyethylene Glycol 6000 | 0.980 |
| Titanium dioxide | 1.400 |
| Talc | 1.200 |
| Iron oxide red | 0.060 |
| Total Film coated tablet | 347.000 |

### EXAMPLE NO. 7

### Composition

| | |
|---|---|
| Constituents | mg/tablet |

Core

| | |
|---|---|
| Flibanserin (free base) | 2.000 |
| Dibasic Calciumphosphate, anhydrous | 61.010 |
| Microcrystalline cellulose | 61.010 |
| HPMC (Methocel E5) | 1.950 |
| Carboxymethylcellulose sodium | 2.600 |
| Colloidal silicon dioxide | 0.650 |
| Magnesium stearate | 0.780 |

Coating

| | |
|---|---|
| HPMC (Methocel E5) | 1.440 |
| Polyethylene Glycol 6000 | 0.420 |
| Titanium dioxide | 0.600 |
| Talc | 0.514 |
| Iron oxide red | 0.026 |
| Total Film coated tablet | 133.000 |

### EXAMPLE NO. 8

### Composition

| | |
|---|---|
| Constituents | mg/tablet |

Core

| | |
|---|---|
| Flibanserin (free base) | 100.000 |
| Dibasic Calciumphosphate, anhydrous | 69.750 |
| Microcrystalline cellulose | 69.750 |
| HPMC (e.g. Methocel E5) | 2.750 |
| Carboxymethylcellulose sodium | 5.000 |
| Colloidal silicon dioxide | 1.250 |
| Magnesium stearate | 1.500 |

Coating

| | |
|---|---|
| HPMC (e.g. Methocel E5) | 2.400 |
| Polyethylene Glycol 6000 | 0.700 |
| Titanium dioxide | 1.043 |
| Talc | 0.857 |
| Total Film coated tablet | 255.000 |

### EXAMPLE NO. 9

### Composition

| | |
|---|---|
| Constituents | mg/tablet |

Core

| | |
|---|---|
| Flibanserin (free base) | 20.000 |
| Lactose monohydrate | 130.000 |
| Microcrystalline cellulose | 43.100 |
| Hydroxypropyl Cellulose (e.g. Klucel LF) | 1.900 |
| Sodium Starch Glycolate | 4.000 |
| Magnesium stearate | 1.000 |

Coating

| | |
|---|---|
| HPMC (e.g. Methocel E5) | 2.400 |
| Polyethylene Glycol 6000 | 0.700 |
| Titanium dioxide | 1.043 |
| Talc | 0.857 |
| Total Film coated tablet | 205.000 |

1. A pharmaceutical compositions comprising a therapeutically effective amount of flibanserin 1 and a therapeutically effective amount of an additional active ingredient 2, selected from the group consisting of compounds used for female hormone replacement therapy 2a, compounds used for menopause problems 2b, and contraceptives 2c.

2. The pharmaceutical compositions according to claim 1, the additional active ingredient 2 is a contraceptive 2c.

3. The pharmaceutical compositions according to claim 2, wherein the contraceptive 2c is selected from chlormadinone acetate, dienogest, drospirenone, etonogestrel, gestodene, medroxyprogesterone acetate, norelgestromine, norethisterone, norethisterone enantate, and norgestimate, optionally in form of the pharmaceutically acceptable salts, in form of the hydrates and/or solvates and optionally in the form of the individual optical isomers, mixtures of the individual enantiomers or racemates thereof.

4. A kit comprising a first pharmaceutical composition comprising a therapeutically effective amount of flibanserin 1, and a second pharmaceutical composition comprising therapeutically effective amount of an active ingredient 2, selected from the group consisting of compounds used for female hormone replacement therapy 2a, compounds used for menopause problems 2b, and contraceptives 2c.

5. (canceled)

6. A method for the treatment of a sexual disorder in a patient in need thereof, comprising administering a therapeutically effective amount of flibanserin 1, in combination with a therapeutically effective amount of an additional active ingredient 2, selected from the group consisting of compounds used for female hormone replacement therapy 2a, compounds used for menopause problems 2b, and contraceptives 2c.

7. The method according to claim 6, wherein the additional active ingredient 2, is a contraceptive 2c.

8. The method according to claim 6, wherein the sexual disorder is Hypoactive Sexual Desire Disorder.

9. The method or use according to one or more of the claim 8, wherein the sexual disorder is Hypoactive Sexual Desire Disorder.
